(19) United States
(12) Patent Application Publication
(10) Pub. No.: US 2013/0296273 A1
(43) Pub. Date: Nov. 7, 2013

(54) TREATMENT OF BLOOD CANCER

(71) Applicant: Karen Curd, Hillsborough, CA (US)

(72) Inventors: John Curd, South San Francisco, CA (US); Damian Handisides, South San Francisco, CA (US); Charles Hart, South San Francisco, CA (US); Stewart Kroll, South San Francisco, CA (US)

(73) Assignee: Threshold Pharmaceuticals, Inc.

(21) Appl. No.: 13/806,088

(22) PCT Filed: Jun. 27, 2011
(86) PCT No.: PCT/US11/42047

§ 371 Date: Jul. 24, 2013

Related U.S. Application Data
(60) Provisional application No. 61/359,313, filed on Jun. 28, 2010.
(60) Provisional application No. 61/470,773, filed on Apr. 1, 2011.

(51) Int. Cl. A61K 31/675; A61K 31/454; A61K 31/573; A61K 31/69

(52) U.S. Cl. CPC A61K 31/675; A61K 31/69; A61K 31/454; A61K 31/573
USPC 514/64; 514/94; 514/99

(57) ABSTRACT

Hypoxia activated prodrugs, such as, e.g., TH-281, TH-302, and TH-308, are useful for the treatment of various blood cancers, such as acute leukemias, chronic leukemias, MDS, MF, and multiple myeloma.

This application claims priority under 35 U.S.C. §119(e) to U.S. provisional application No. 61/359,313 filed on Jun. 28, 2010 and to U.S. provisional application No. 61/470,773 filed on Apr. 1, 2011, each of which is incorporated herein in its entirety by reference.

### FIELD OF THE INVENTION

The present invention relates to treatment of blood cancer by the administration of hypoxia activated prodrugs and so relates to the field of cellular biology, medicinal chemistry, medicine, molecular biology, and pharmacology.

### BACKGROUND OF THE INVENTION

Blood cancer refers to a class of cancers that attack the blood, bone marrow, and/or lymphatic system. This class of cancers includes leukemia and multiple myeloma, all of which can be life-threatening diseases for which new and more efficacious treatments are needed.

The relation between cancer cells and the tumor microenvironment affects the growth and survival of cancer cells (see Hiruma et al., Blood. 2009; 113(20): 4894-4902, and Podar et al., Leukemia. 2009; 23(1): 10-24, each of which is incorporated herein by reference). Hypoxia, or low oxygen level, is a characteristic of the microenvironment of many solid tumors and results from the poor vascularization that characterizes many solid tumors. High metastatic potential and poor prognosis correlate highly with hypoxia in solid tumors.

Clinical research has attempted to target therapies to the hypoxic regions of solid tumors for many years without notable success. Recently, however, a promising new class of hypoxia-activated prodrugs has emerged (see U.S. Pat. No. 7,550,496, incorporated herein by reference), and the most promising compound in that class, called TH-302 (see PCT Pub. Nos. 2007/002931; 2008/083101; and 2010/048330, each of which is incorporated herein by reference), is now in advanced clinical testing.

Given the prominent role, however, of the circulatory system in oxygenating tissues and the lack of clinical success with earlier hypoxia-targeted therapies, there has been no appreciation that hypoxia-targeted therapies might meet the need for new blood cancer therapies. The present invention meets that need.

### SUMMARY

Provided herein are methods for treating various blood cancers, such as acute leukemias (AML and ALL), chronic leukemias (CML and CLL), idiopathic myelofibrosis (MF, also known as agnogenic myeloid metalplasia or AMM), lymphoma, myelodysplastic syndrome (MDS), and multiple myeloma, comprising administering a therapeutically effective amount of a hypoxia activated prodrug, including but not limited to a compound of formula (I):

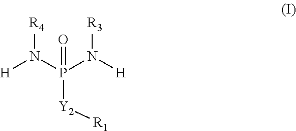

wherein Y₂ is O, S, NR₆, NCOR₆, or NSO₂R₆ wherein R₆ is (C₁-C₆) alkyl, C₁-C₆ heteroalkyl, aryl, or heteroaryl; R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl; R₁ has the formula L-Z₃; L is C(Z₁)₂; each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl; or L is:

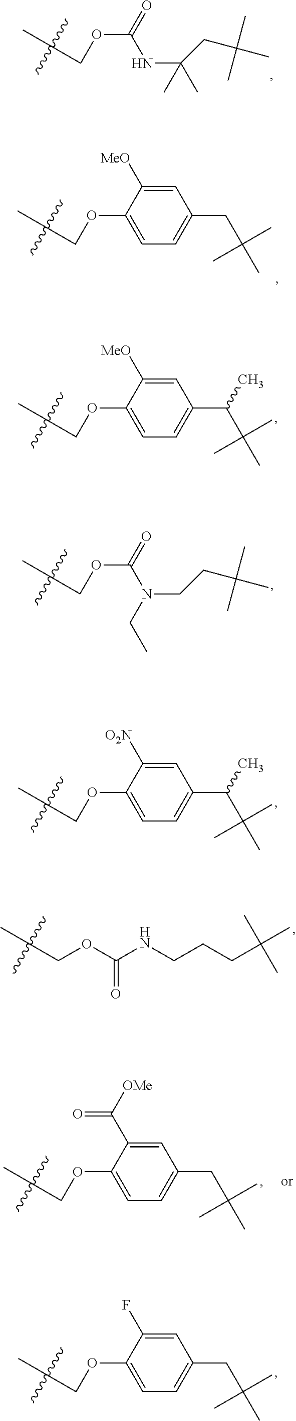

Z₃ is a bioreductive group having a formula selected from the group consisting of:

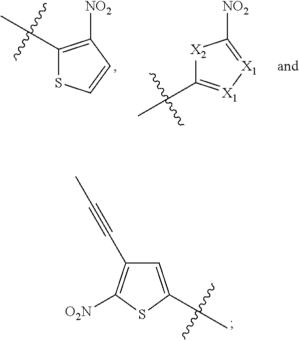

wherein each X₁ is independently N or CR₈; X₂ is NR₇, S, or O; each R₇ is independently C₁-C₆ alkyl, C₁-C₆ heteroalkyl, C₃-C₈ cycloalkyl, heterocyclyl, aryl or heteroaryl; and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl; or a pharmaceutically acceptable salt thereof. In various embodiments of the invention, the compound utilized in this invention is a compound of formula I that is TH-281, TH-302, or TH-308 (structures provided below).

In one embodiment, TH-302 or another compound of formula I is administered as single agent (“agent” is used interchangeably with “drug” herein) therapy to treat a blood cancer selected from the group consisting of AML, ALL, CML, CLL, MDS, and MF, including relapsed or refractory forms of these cancers. In one embodiment, TH-302 or another compound of formula I is administered for five consecutive days of a 21 day cycle, and the dose is between 120 and 575 mg/m²/day, including, for example, doses of 180, 240, 340, and 480 mg/m²/day. In some embodiments, the five daily doses are spread out over 8 days. In one embodiment, pimonidazole is used as a marker of hypoxia and infused over twenty minutes at a dosage of 0.5 g/m² dissolved in 0.9% saline approximately sixteen (±6) hours prior to a bone marrow biopsy. The maximum dose of pimonidazole can be held to 1.0 grams for patients with a body surface area (BSA) >2.0 m².

These methods of the invention have been demonstrated to be efficacious in clinical testing. Two AML patients treated with TH-302 at the 120 mg/m²/day dose had stable disease. One patient with ALL treated with TH-302 at the 170 mg/m²/day dose had a partial response by bone marrow biopsy after cycle 1. Two patients (one with AML and one with ALL) treated with TH-302 at the 240 mg/m²/day dose and one patient treated with TH-302 at the 330 mg/m²/day had stable disease after cycle 1.

In one embodiment, TH-302 or another compound of formula I is administered as single agent therapy to treat multiple myeloma, including relapsed or refractory forms of this disease, including but not limited to patients who have failed bortezomib and/or lenalidomide (or thalidomide) therapy. In one embodiment, TH-302 or another compound of formula I is administered on days 1, 4, 8, and 11 of a 21 day cycle, and the dose is between 120 and 575 mg/m²/day, including, for example, doses of 180, 240, 340, and 480 mg/m²/day.

In one embodiment, TH-302 or another compound of formula I is administered as a combination therapy with bortezomib to treat multiple myeloma, including relapsed or refractory forms of this disease, in patients, including but not limited to those, who have failed bortezomib and/or lenalidomide (or thalidomide) therapy. In one embodiment, TH-302 or another compound of formula I is administered on days 1, 4, 8, and 11 of a 21 day cycle, and the dose is between 120 and 575 mg/m²/day, including, for example, doses of 180, 240, 340, and 480 mg/m²/day. The bortezomib is administered as commercially supplied and approved at a dose of 1.3 mg/m²/day or at a dose of 1.0 mg/m²/day on the same cycle. In one embodiment, TH-302 or another compound of formula I is administered at least 2 hours before the bortezomib. See PCT Pub. No. 2010/048330, incorporated herein by reference.

In one embodiment, TH-302 or another compound of formula I is administered as a combination therapy with lenalidomide and dexamethasone to treat multiple myeloma, including relapsed or refractory forms of this disease, in patients including but not limited to those, who have failed bortezomib and/or lenalidomide (or thalidomide) therapy. In one embodiment, TH-302 or another compound of formula I is administered on days 1, 4, 8, and 11 of a 21 day cycle, and the dose is between 120 and 575 mg/m²/day, including, for example, doses of 180, 240, 340, and 480 mg/m²/day. The lenalidomide is administered as commercially supplied and approved at a dose of 25 mg on days 1 to 14 and dexamethasone is administered as commercially supplied and approved at a dose of 40 mg on days 1-4 and 9-12 of the same cycle. In one embodiment, TH-302 or another compound of formula I is administered on days 1, 4, 8, 11, 15, 18 of a 28 day cycle, and the dose is between 120 and 575 mg/m²/day, including, for example, doses of 180, 240, 340, and 480 mg/m²/day. The lenalidomide is administered as commercially supplied and approved at a dose of 25 mg on days 1 to 21 and dexamethasone is administered as commercially supplied and approved at a dose of 40 mg on days 1-4, 9-12 and 17-20 of the same 28 day cycle.

In one embodiment, TH-302 or another compound of formula I is provided in 100 mg vials, lyophilized, and dissolved in (dextrose 5% in water) D5W and administered intravenously over approximately 30-60 minutes via an infusion pump. The infusion volume depends on the total dose given (in mg) during the infusion. If <1000 mg is being infused, 500 cc of D5W are used for infusion. If the total dose is >1000, 1000 cc of D5W are used for infusion.

In various embodiments of the invention, a biomarker of hypoxia is used to select patients for treatment and/or to identify patients that are responding to therapy.

These and other aspects and embodiments of the invention are described in additional detail below.

### DETAILED DESCRIPTION

### Definitions

In this specification and in the claims that follow, reference will be made to a number of terms that shall be defined to have the meanings below. All numerical designations, e.g., pH, temperature, time, concentration, and weight, including ranges, are approximations that typically may be varied (+) or (−) by increments of 0.1, 1.0, or 10.0, as appropriate. All numerical designations may be understood as preceded by the term “about”. Reagents described herein are exemplary and equivalents of such may be known in the art.

The singular form “a”, “an”, and “the” includes plural references unless the context clearly dictates otherwise.

The term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

Certain terms related to formula I are defined below.

“Acyl” refers to —CO-alkyl, wherein alkyl is as defined here.

“Aroyl” refers to —CO-aryl, wherein aryl is as defined here.

“Alkoxy” refers to —O-alkyl, wherein alkyl is as defined here.

“Alkenyl” refers to a linear monovalent hydrocarbon radical or a branched monovalent hydrocarbon radical having the number of carbon atoms indicated in the prefix and containing at least one double bond, but no more than three double bonds. For example, (C₂ -C₆)alkenyl includes, ethenyl, propenyl, 1,3-butadienyl and the like. Alkenyl can be optionally substituted with substituents, including for example, deuterium (“D”), hydroxyl, amino, mono or di(C₁-C₆)alkyl amino, halo, C₂ -C₆ alkenyl ether, cyano, nitro, ethynyl, C₁-C₆ alkoxy, C₁-C₆ alkylthio, —COON, —CONH₂, mono- or di(C₁-C₆)alkylcarboxamido, —SO₂NH₂, —OSO₂—(C₁-C₆)alkyl, mono or di(C₁-C₆) alkylsulfonamido, aryl, heteroaryl, alkyl or heteroalkylsulfonyloxy, and aryl or heteroarylsulfonyloxy.

“Alkyl” refers to a linear saturated monovalent hydrocarbon radical or a branched saturated monovalent hydrocarbon radical having the number of carbon atoms indicated in the prefix. As used in this disclosure, the prefixes (C₁-C_{qq}), C_{1-qq}, or C₁-C_{qq}, wherein qq is an integer from 2-20, have the same meaning. For example, (C₁-C₆)alkyl, C₁₋₆ alkyl, or C₁-C₆ alkyl includes methyl, ethyl, n-propyl, 2-propyl, n-butyl, 2-butyl, tert-butyl, pentyl, and the like. For each of the definitions herein (e.g., alkyl, alkenyl, alkoxy, etc.), when a prefix is not included to indicate the number of main chain carbon atoms in an alkyl portion, the radical or portion thereof will have six or fewer main chain carbon atoms. (C₁ -C₆)alkyl can be optionally substituted with substituents, including for example, deuterium (“D”), hydroxyl, amino, mono or di(C₁-C₆) alkyl amino, halo, C₂-C₆ alkenyl ether, cyano, nitro, ethenyl, ethynyl, C₁-C₆ alkoxy, C₁-C₆ alkylthio, —COON, —CONH₂, mono- or di(C₁-C₆)alkylcarboxamido, —SO₂NH₂, —OSO₂—(C₁-C₆)alkyl, mono or di(C₁-C₆) alkylsulfonamido, aryl, heteroaryl, alkylsulfonyloxy, heteroalkylsulfonyloxy, arylsulfonyloxy or heteroarylsulfonyloxy.

“Alkylamino” or mono-alkylamino refers to —NH-alkyl, wherein alkyl is as defined here.

“Alkynyl” refers to a linear monovalent hydrocarbon radical or a branched monovalent hydrocarbon radical having the number of carbon atoms indicated in the prefix and containing at least one triple bond, but no more than two triple bonds. For example, (C₂-C₆)alkynyl includes, ethynyl, propynyl, and the like. Alkynyl can be optionally substituted with substituents, including for example, deuterium (“D”), hydroxyl, amino, mono or di(C₁-C₆)alkyl amino, halo, C₂-C₆ alkenyl ether, cyano, nitro, ethenyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆)alkylsulfonamido, aryl, heteroaryl, alkyl or heteroalkylsulfonyloxy, and aryl or heteroarylsulfonyloxy.

“Aryl” refers to a monovalent monocyclic or bicyclic aromatic hydrocarbon radical of 6 to 10 ring atoms which is substituted independently with one to eight substituents, preferably one, two, three, four of five substituents selected from deuterium (“D”), alkyl, cycloalkyl, cycloalkylalkyl, halo, nitro, cyano, hydroxyl, alkoxy, amino, acylamino, mono-alkylamino, di-alkylamino, haloalkyl, haloalkoxy, heteroalkyl, COR (where R is hydrogen, alkyl, cycloalkyl, cycloalkyl-alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl) or —(CR′R″)ₙ—CONR^{x}R^{y} (where n is an integer from 0 to 5, R′ and R″ are independently hydrogen or alkyl, and R^{x} and R^{y} are independently selected from hydrogen, alkyl, cycloalkyl, cycloalkylalkyl, phenyl or phenylalkyl). In one embodiment, R^{x} and R^{y} together is cycloalkyl or heterocyclyl. More specifically the term aryl includes, but is not limited to, phenyl, biphenyl, 1-naphthyl, and 2-naphthyl, and the substituted forms thereof.

“Cycloalkyl” refers to a monovalent cyclic hydrocarbon radical of three to seven ring carbons. The cycloalkyl group can have one or more double bonds and can also be optionally substituted independently with one, two, three or four substituents selected from alkyl, optionally substituted phenyl, or —C(O)R^{z} (where R^{z} is hydrogen, alkyl, haloalkyl, amino, mono-alkylamino, di-alkylamino, hydroxyl, alkoxy, or optionally substituted phenyl). More specifically, the term cycloalkyl includes, for example, cyclopropyl, cyclohexyl, cyclohexenyl, phenylcyclohexyl, 4-carboxycyclohexyl, 2-carboxamidocyclohexenyl, 2-dimethylaminocarbonylcyclohexyl, and the like.

“Dialkylamino” or di-alkylamino refers to —N(alkyl)₂, wherein alkyl is as defined here.

“Heteroalkyl” refers to an alkyl radical as defined herein with one, two or three substituents independently selected from cyano, —OR^{w}, —NR^{x}R^{y}, and —S(O)ₚR^{z} (where p is an integer from 0 to 2), with the understanding that the point of attachment of the heteroalkyl radical is through a carbon atom of the heteroalkyl radical. R^{w} is hydrogen, alkyl, cycloalkyl, cycloalkyl-alkyl, aryl, aralkyl, alkoxycarbonyl, aryloxycarbonyl, carboxamido, or mono- or di-alkylcarbamoyl. R^{x} is hydrogen, alkyl, cycloalkyl, cycloalkyl-alkyl, aryl or araalkyl. R^{y} is hydrogen, alkyl, cycloalkyl, cycloalkyl-alkyl, aryl, araalkyl, alkoxycarbonyl, aryloxycarbonyl, carboxamido, mono- or di-alkylcarbamoyl or alkylsulfonyl. R^{z} is hydrogen (provided that n is 0), alkyl, cycloalkyl, cycloalkyl-alkyl, aryl, araalkyl, amino, mono-alkylamino, di-alkylamino, or hydroxyalkyl. Representative examples include, for example, 2-hydroxyethyl, 2,3-dihydroxypropyl, 2-methoxyethyl, benzyloxymethyl, 2-cyanoethyl, and 2-methylsulfonyl-ethyl. For each of the above, R^{w}, R^{x}, R^{y}, and R^{z} can be further substituted by amino, halo, fluoro, alkylamino, di-alkylamino, OH or alkoxy. Additionally, the prefix indicating the number of carbon atoms (e.g., C₁-C₁₀) refers to the total number of carbon atoms in the portion of the heteroalkyl group exclusive of the cyano, —OR^{w}, —NR^{x}R^{y}, or —S(O)ₚR^{z} portions. In one embodiment, R^{x} and R^{y} together is cycloalkyl or heterocyclyl.

“Heteroaryl” refers to a monovalent monocyclic, bicyclic or tricyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring is optionally substituted independently with one to eight substituents, preferably one, two, three or four substituents, selected from alkyl, cycloalkyl, cycloalkyl-alkyl, halo, nitro, cyano, hydroxyl, alkoxy, amino, acylamino, mono-alkylamino, di-alkylamino, haloalkyl, haloalkoxy, heteroalkyl, —COR (where R is hydrogen, alkyl, phenyl or phenylalkyl, —(CR′R″)ₙ—COOR (where n is an integer from 0 to 5, R′ and R″ are independently hydrogen or alkyl, and R is hydrogen, alkyl, cycloalkyl, cycloalkyl-alkyl, phenyl or phenylalkyl), or —(CR′R″)ₙ—CONR^{x}R^{y} (where n is an integer from 0 to 5, R′ and R″ are independently hydrogen or alkyl, and R^{x} and R^{y} are, independently of each other, hydrogen, alkyl, cycloalkyl, cycloalkyl-alkyl, phenyl or phenylalkyl). In one embodiment, R^{x} and R^{y} together is cycloalkyl or heterocyclyl. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, indazolyl, pyrrolopyrymidinyl, indolizinyl, pyrazolopyridinyl, triazolopyridinyl, pyrazolopyrimidinyl, triazolopyrimidinyl, pyrrolotriazinyl, pyrazolotriazinyl, triazolotriazinyl, pyrazolotetrazinyl, hexaaza-indenly, and heptaaza-indenyl and the derivatives thereof. Unless indicated otherwise, the arrangement of the hetero atoms within the ring can be any arrangement allowed by the bonding characteristics of the constituent ring atoms.

“Heterocyclyl” or “cycloheteroalkyl” refers to a saturated or unsaturated non-aromatic cyclic radical of 3 to 8 ring atoms in which one to four ring atoms are heteroatoms selected from O, NR (where R is hydrogen, alkyl, cycloalkyl, cycloalkylalkyl, phenyl or phenylalkyl), P(═O)OR^{w}, or S(O)ₚ (where p is an integer from 0 to 2), the remaining ring atoms being C, wherein one or two C atoms can optionally be replaced by a carbonyl group. The heterocyclyl ring can be optionally substituted independently with one, two, three or four substituents selected from alkyl, aryl, arylalkyl, heteroaryl, heteroarylalkyl, cycloalkyl, cycloalkylalkyl, halo, nitro, cyano, hydroxyl, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl), —(CR′R″)ₙ—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically the term heterocyclyl includes, but is not limited to, pyridyl, tetrahydropyranyl, N-methylpiperidin-3-yl, N-methylpyrrolidin-3-yl, 2-pyrrolidon-1-yl, furyl, quinolyl, thienyl, benzothienyl, pyrrolidinyl, piperidinyl, morpholinyl, pyrrolidinyl, tetrahydrofuranyl, tetrahydrothiofuranyl, 1,1-dioxo-hexahydro-1Δ⁶-thiopyran-4-yl, tetrahydroimidazo[4,5-c]pyridinyl, imidazolinyl, piperazinyl, and piperidin-2-only and the derivatives thereof. The prefix indicating the number of carbon atoms (e.g., C₃-C₁₀) refers to the total number of carbon atoms in the portion of the cycloheteroalkyl or heterocyclyl group exclusive of the number of heteroatoms.

“Heteroacyl” refers to —CO-heteroalkyl, wherein heteroalkyl is as defined here.

“Heteroaroyl” refers to —CO-heteroayl, wherein heteroaryl is as defined here.

“Rₛᵤₗ sulfonyloxy” refers to Rₛᵤₗ —S(═O)₂—O— and includes alkylsulfonyloxy, heteroakylsulfonyloxy, cycloalkylsulfonyloxy, heterocyclylsulfonyloxy, arylsulfonyloxy and heteroarylsulfonyloxy wherein Rₛᵤₗ is alkyl, heteroakyl, cycloalkyl, heterocyclyl, aryl and heteroaryl respectively, and wherein alkyl, heteroakyl, cycloalkyl, heterocyclyl, aryl and heteroaryl are as defined here. Examples of alkylsulfonyloxy include Me-S(═O)₂—O—, Et-S(═O)₂—O—, CF₃—S(═O)₂—O— and the like, and examples of arylsulfonyloxy include:

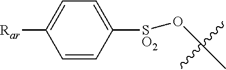

wherein Rₐᵣ is H, methyl, or bromo.

“Substituents” refers to, along with substituents particularly described in the definition of each of the groups above, those selected from: deuterieum, -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH)═NH, —NH—C(NH₂)═NR′, —S(O)R′, —S(O)₂R′, —S(O)₂NR′R″, —NR′S(O)₂R″, —CN, —NO₂, —R′, —N₃, perfluoro(C₁-C₄)alkoxy, and perfluoro(C₁-C₄)alkyl, in a number ranging from zero to the total number of open valences on the radical; and where R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl, aryl substituted with 1-3 halogens, unsubstituted C₁₋₈ alkyl, C₁₋₈alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. Other suitable substituents include each of the above aryl substituents attached to a ring atom by an alkylene tether of from 1-4 carbon atoms. Two of the substituents on adjacent atoms of the aryl or heteroaryl ring may optionally be replaced with a substituent of the formula -T²-C(O)—(CH₂)_{q}-—U³—, wherein T² and U³ are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ-B-, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X⁵—(CH₂)ₜ—, wherein s and t are independently integers of from 0 to 3, and X⁵ is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

Certain compounds utilized in the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example, and without limitation, tritium (³H), iodine-125 (¹²⁵I) or carbon-14 (¹⁴C). All isotopic variations of the compounds of the present invention, whether radioactive or not, are intended to be encompassed within the scope of the present invention.

Other terms related to this invention are defined below.

“Acute” in the context of blood cancers, refers to the relatively short time course in which these cancers can become extremely serious and even lead to the death of a patient (e.g., they can be fatal in as little as a few weeks if left untreated) and differentiates them from “chronic” blood cancers, which may not have extremely debilitating effects on or lead to the death of a patient for many years. “Acute leukemias” refer to ALL, AML, and the like. “Chronic leukemias” refer to CLL, CML, myelofibrosis, and the like.

“Acute Lymphoblastic Leukemia (ALL)” refers to a blood cancer, particularly a cancer affecting the white blood cells, and is characterized by hyperproliferation of lymphoblasts. In ALL, malignant, immature white blood cells continuously multiply and are overproduced in the bone marrow. ALL cells crowd out normal cells in the bone marrow and may metastasize to other organs. ALL is also known as acute lymphocytic leukemia and acute childhood leukemia.

“Acute Myeloid (Myelogenous) Leukemia (AML)” refers to a blood cancer in which white blood cells known as “myeloid cells” become cancerous. In AML, the bone marrow produces abnormal blood cells called “myeloblasts,” leading to the replacement of normal blood cells with abnormal cells and disrupting the normal function of the bone marrow. With the abnormal production of “blast” cells, the production of normal blood marrow cells is inhibited, causing a deficiency of red blood cells, normal white blood cells, and platelets, leading to deleterious effects such as anemia, vulnerability to bruising and bleeding, and increased risk of infection.

“Administering” or “administration of” a drug to a patient (and grammatical equivalents of this phrase) refer both to direct administration, which may be administration to a patient by a medical professional or may be self-administration, as well as to indirect administration, which may be the act of prescribing a drug. For example, a physician who instructs a patient to self-administer a drug and/or provides a patient with a prescription for a drug is administering the drug to the patient.

“Blood cancer” refers to a hematological malignancy involving abnormal hyperproliferation or malignant growth and/or metastasis of a blood cell. Blood cancers include, without limitation, acute leukemias (AML and ALL), chronic leukemias (CML and CLL), idiopathic myelofibrosis (MF, also known as agnogenic myeloid metaplasia or AMM), lymphoma, myelodysplastic syndrome (MDS), and multiple myeloma.

“Bone marrow stem cell transplant” refers to replacing a patient's bone marrow with new bone marrow. In these transplants, chemotherapy drugs are used to kill the stem cells in the bone marrow (including those creating diseased lymphocytes), and then, healthy adult blood stem cells from a donor (allogenic transplant) or from the patient's own bone marrow (autologus transplant) are infused into the blood, wherein they travel to the bone marrow and begin making healthy blood cells.

“Chronic lymphocytic (or lymphoid) leukemia (CLL)” refers to a blood cancer affecting B cell lymphocytes. B cells originate in the bone marrow and develop in the lymph nodes. In CLL, the B cells grow in an uncontrolled manner and accumulate in the bone marrow and blood, wherein they crowd out healthy blood cells. As the disease advances, CLL results in swollen lymph nodes, spleen, and liver.

“Chronic myelogenous leukemia (CML)” refers to a blood cancer in which the bone marrow produces granulocytes, some of which never mature into white blood cells. The “immature” white blood cells are called “blasts.” Over time, the granulocytes and blasts grow out of control and result in a platelet and red blood cell deficiency in the bone marrow. CML patients may have a gene mutation called the “Philadelphia chromosome.” This chromosome causes the bone marrow to make certain tyrosine kinases that result in the development of granulocytes or blasts. Some CML patients have a form of the disease resistant to treatment with tyrosine kinase inhibitors. CML includes, without limitation, chronic myelogenous leukemia, chronic myeloid leukemia, chronic myelocytic leukemia, and chronic granulocytic leukemia (CGL).

“Combination therapy” refers to the use of two or more drugs in therapy, i.e., use of a hypoxia activated prodrug as described herein together with conventional drugs used to treat blood cancer is a combination therapy. Administration in “combination” refers to the administration of two agents (e.g., a hypoxia activated prodrug and an agent known for treating a blood cancer) in any manner in which the pharmacological effects of both manifest in the patient at the same time. Thus, administration in combination does not require that a single pharmaceutical composition, the same dosage form, or even the same route of administration be used for administration of both agents or that the two agents be administered at precisely the same time. For example, and without limitation, it is contemplated that one or more of the following agents can be administered in combination with a hypoxia activated prodrug in accordance with the present invention: alemtuzumab (Campath®, Genzyme), amsacrine, asparaginase (also called crisantaspase), bendamustine, bortezomib, busulfan, carmustine, chlorambucil, cyclophosphamide, cytarabine (ara-C), daunorubicin, dexamethasone, doxorubicin (Adriamycin®, Bedford Laboratories), etoposide, fludarabine, hydroxyurea, hypomethylating agents, including but not limited to, azacytidine and decitabine, idarubicin, immunomodulating agents including, without limitation, lenalidomide and thalidomide, immunosuppression agents, including without limitation, anti-thymocyte globulin (ATG) and cyclosporine, interferon-α 2b, mercaptopurine (6-MP), melphalan, methotrexate, ofatumumab (Arzerra®, GlaxoSmithKline and Genmab), prednisone, rituximab (Rituxan®, Genentech), teniposide, thalidomide, thioguanine, topotecan, tyrosine kinase inhibitors, including but not limited to imatinib, dasatinib, and nilotinib, and vincristine.

“Hypoxia activated prodrug” refers to a drug that is less active or inactive under normoxia than under hypoxia or anoxia. Hypoxia activated prodrugs include drugs that are activated by a variety of reducing agents and reducing enzymes, including without limitation single electron transferring enzymes (such as cytochrome P450 reductases) and two electron transferring (or hydride transferring) enzymes (see US Pat. App. Pub. Nos. 2005/0256191, 2007/0032455, and 2009/0136521, and PCT Pat. App. Pub. Nos. WO 2000/064864, WO 2004/087075, and WO 2007/002931, each of which is incorporated herein by reference). The hypoxia activated prodrugs useful in the methods of the present invention are compounds of formula I, including but not limited to compounds where Z₃, as defined by that formula, is a 2-nitroimidazole moiety. Examples of particular hypoxia activated prodrugs useful in the methods of the invention include without limitation TH-281, TH-302, and TH-308. Methods of synthesizing and formulating TH-302 and other compounds of formula I are described in PCT Pub. Nos. WO 2007/002931 and WO 2008/083101, each of which is incorporated herein by reference.

“Multiple myeloma (MM)” refers to a blood cancer having clonal B cell malignancy characterized by the accumulation of neoplastic plasma cells in the bone marrow. There are several types of multiple myeloma, including smoldering multiple myeloma (SMM), plasma cell leukemia, nonsecretory myeloma, osteosclerotic myeloma (POEMS syndrome), solitary plasmacytoma (also called solitary myeloma of the bone), and extramedullary plasmacytoma.

“Myelodysplastic syndrome (MDS)” refers to a blood cancer that occurs when the bone marrow stops producing healthy blood cells and instead produces immature blood cells that function poorly. This results in the production of too many defective blood cells and not enough healthy blood cells. In people with MDS, the disorder begins when a defect occurs in a stem cell in the bone marrow. That stem cell, in turn, produces blood cells that carry the same defect. These defective cells grow to outnumber healthy blood cells and live longer. These defective cells may also kill other stem cells too early, resulting in low blood counts. The abnormal cells also crowd out the healthy cells. MDS can progress over time into acute myelogenous leukemia.

“Myelofibrosis” refers to a type of chronic leukemia that disrupts the body's normal production of blood cells. Myelofibrosis can occur on its own (primary myelofibrosis) or it can occur as a result of another bone marrow disorder (secondary myelofibrosis). Advanced myelofibrosis gets progressively worse and can eventually develop into a more serious form of leukemia.

“Patient” or “subject” refers to mammals, particularly humans, but also to animals such as simians, cattle, horses, dogs, cats, and rodents suffering from blood cancer.

“Relapsed or refractory” refers to a type of blood cancer that is resistant to treatment with an agent, or responds to treatment with an agent but comes back without being resistant to that agent, or responds to treatment with an agent but comes back resistant to that agent.

“Single agent therapy” or “monotherapy” refers to using a single drug to treat a disease, i.e., using a hypoxia activated prodrug such as, for example, TH-302 as the only chemical agent to treat a blood cancer. Administration of palliatives and/or vitamins and/or other agents that are administered for purposes other than to treat directly the disease can be administered in single agent therapy. A patient undergoing single agent therapy may also undergo radiation therapy and/or surgery.

“Standard chemotherapy” refers to treatment with drugs in accordance with FDA labeling instructions and/or good clinical practice. Standard chemotherapy is well known to those of skill in the medical arts.

“TH-281” refers to the compound of formula:

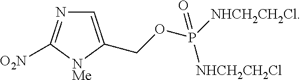

“TH-302” refers to the compound of formula:

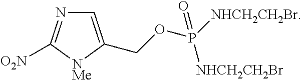

“TH-308” refers to the compound of formula:

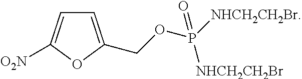

“Therapeutically effective amount” of a drug or an agent refers to an amount of drug or agent that, when administered to a patient with blood cancer, will have the intended therapeutic effect, e.g., alleviation, amelioration, palliation or elimination of one or more manifestations of the blood cancer in the patient. A therapeutic effect does not necessarily occur by administration of one dose and may occur only after administration of a series of therapeutically effective doses. Thus, a therapeutically effective amount may be administered in one or more administrations.

“Treating” or “treatment of” a condition or patient refers to taking steps to obtain beneficial or desired results, including clinical results. For purposes of this technology, beneficial or desired clinical results include, but are not limited to, alleviation or amelioration of one or more symptoms of blood cancer; diminishment of extent of disease; delay or slowing of disease progression; amelioration, palliation, or stabilization of the disease state; or other beneficial results.

### Treatment Methods

Hypoxia may be relevant for normal marrow hematopoiesis, the formation of blood cells from hematopoietic stem cells (Lennon et al., J. Cell Physiol., 2001;187(3):345-355; Morrison et al., J Neurosci., 2000;20(19):7370-7376; and Parmar et al., Proc Natl Acad Sci USA. 2007;104(13):5431-5436, each of which is incorporated herein by reference). The present invention arose in part from the discovery that hypoxia is relevant in the etiology and pathogenesis of abnormal hematopoiesis, but that administration of hypoxia activated prodrugs of formula I could target that abnormal hematopoiesis selectively, providing a new treatment for blood cancers such as leukemias, lymphomas, and multiple myeloma.

In one aspect, the present invention provides a method of treating a blood cancer comprising administering a therapeutically effective amount of a hypoxia activated prodrug of formula I. In various embodiments, the hypoxia activated prodrug is selected from the group consisting of TH-281, TH-302, and TH-308. In one important embodiment, the prodrug is TH-302. The hypoxia activated prodrug is administered in a therapeutically effective amount to a patient in need of such treatment, thereby treating the blood cancer. Illustrative blood cancers amenable to treatment include those selected from multiple myeloma, an acute leukemia, a chronic leukemia, an advanced phase chronic myelogenous leukemia (CML), myelodysplastic syndrome, a high risk MDS, MF, an advanced myelofibrosis, a chronic lymphocytic leukemia (CLL), and a relapsed or refractory form of any of the foregoing.

In one embodiment, the hypoxia activated prodrug administered is TH-302. In one embodiment, TH-302 or another compound of formula I is administered as a 30 minute intravenous infusion daily for 5 days every 21 days. In one embodiment, TH-302 or another compound of formula I is administered in a one week dosing cycle including once daily administration for 5 days followed by 2 days of no TH-302 administration. In one embodiment, TH-302 or another compound of formula I is administered for 3 or more such cycles. In various embodiments, TH-302 or another compound of formula I is administered for up to about 25 or up to about 50 such cycles. In one embodiment, the therapeutically effective amount is a daily dose of about 120 mg/m² to about 460 mg/m². In one embodiment, TH-302 or another compound of formula I is administered once weekly. In one embodiment, the therapeutically effective amount is a once weekly dose of about 575 mg/m² to about 670 mg/m². In one embodiment, the therapeutically effective amount is a once weekly dose of about 240 mg/m² administered in 3 week cycles. In various embodiments, the therapeutically effective amount is a daily dose of about 240 mg/m² to about 480 mg/m² administered on days 1 and 8 of a 3 week cycle that may be repeated one, two, three, or more times.

In another embodiment, in accordance with the methods of the present technology for treating blood cancers, TH-302 or another compound of formula I is administered in combination with another anti cancer agent. In one embodiment, the other anti cancer drug is Velcade® (bortezomib, Millennium Pharmaceuticals) or lenalidomide (Revlimid®, Cellgene). When administered in combination with another anti cancer drug, in one embodiment, TH-302 or another compound of formula I is administered before administering the other anti cancer drug. For example, TH-302 may be administered the day before the other anti cancer drug is administered or, if the two drugs are administered on the same day, then TH-302 or another compound of formula I may be administered from at least 30 minutes to up to 4 hours or even 8 hours before the other anti cancer drug. See PCT Pub. No. WO 2008/083101, incorporated herein by reference. In various embodiments, TH-302 or another compound of formula I is administered as a monotherapy for 1 for 2 cycles before combination therapy is initiated by administering a second anti cancer drug. In various embodiments of mono- and combination therapies, each cycle of TH-302 or another compound of formula I administration is a one week cycle including once daily administration for 5 days followed by 2 days of no TH-302 administration.

In various embodiments, a method of the invention is employed as a first, second, third or later line of treatment. As used herein, a “first line” or “second line” or “third line” of treatment refers to a place in the order of treatment with different medications or other therapies received by a patient. First line therapy regimens are treatments given first, whereas second or third line therapy are given after the first line therapy or after the second line therapy, respectively. Therefore, first line therapy is “the first treatment for a disease or condition.” In patients with cancer, first line therapy, sometimes referred to as “primary therapy” or “primary treatment”, can be surgery, chemotherapy, radiation therapy, or a combination of these therapies. Typically, a patient is given a subsequent chemotherapy regimen (second or third line therapy), either because the patient did not show a positive clinical or showed only a sub-clinical response to a first or second line therapy or showed a positive clinical response but later experienced a relapse, sometimes with disease now resistant to the earlier therapy that elicited the earlier positive response.

In one embodiment, TH-302 or another compound of formula I is administered as single agent therapy to treat a blood cancer, including relapsed or refractory forms of these cancers. In one embodiment, the TH-302 or another compound of formula I is administered for five consecutive days of a 21 day cycle, and the dose is between 120 and 575 mg/m²/day, including, for example, doses of 180, 240, 340, and 480 mg/m²/day. In some embodiments, the five daily doses are spread out over 8 days. In one embodiment, pimonidazole or another suitable marker of hypoxia is used as a marker of hypoxia and infused over twenty minutes at a dosage of 0.5 g/m² dissolved in 0.9% saline approximately sixteen (±6) hours prior to a bone marrow biopsy. The maximum dose of pimonidazole can be held to 1.0 grams for patients with a BSA >2.0.

These methods of the invention have been demonstrated to be efficacious in ALL and AML. Two patients with AML treated with TH-302 at the 120 mg/m²/day dose had stable disease. One patient with ALL treated with TH-302 at the 170 mg/m²/day dose had a partial response based on the normalization of blast count as measured by bone marrow biopsy after cycle 1. One patient with AML and one patient with ALL treated with TH-302 at the 240 mg/m²/day dose had stable disease after cycle 1. One patient with ALL treated with TH-302 at the 330 mg/m²/day dose had stable disease after cycle 1.

In one embodiment, a patient treated in accordance with the present invention is selected for treatment comprising administering a hypoxia activated prodrug, alone or in combination with another agent, based on the patient having the Philadelphia chromosome; in another embodiment, such a patient is diagnosed as having a chronic leukemia such as CML or an acute leukemia such as ALL or AML. Suitable methods for detecting Philadelphia chromosome are well known to one of skilled in the art. See, e.g., Sawyers, The New England Journal of Medicine, 1999, 340(17): 1330-40, incorporated herein by reference. In another embodiment, the the selected patient is administered a therapeutically effective amount of the hypoxia activated prodrug to the cancer patient selected thereby treating the cancer. Blood cancer treatment methods as disclosed herein are useful for such treatment. In one embodiment, the hypoxia activated prodrug administered is TH-302.

Methods of preparation of and pharmaceutical compositions of hypoxia activated prodrugs, and other methods of treating cancer by administering various hypoxia activated prodrugs of formula I are described in Duan et al., J. Med. Chem. 2008, 51, 2412-2420 PCT Pub. Nos. 2007/002931, 2008/083101, and 2010/048330, each of which is incorporated herein by reference. Other methods of treating blood cancers, which may be used in combination with the methods of the present invention, are known to one of skilled in the art, and are described, for example, in the product descriptions found in the 2010 or more current edition of the Physician's Desk Reference, Medical Economics Company, Inc., Oradell, N.J.; Goodman and Gilman's The pharmacological basis of therapeutics., Eds. Hardman et al., McGraw-Hill. New York. (US) 2011, 12th Ed., and in publications of the U.S. Food and Drug Administration and the NCCN Guidelines (National Comprehensive Cancer Network). Such described and known methods can be appropriately modified by one of skill in the art, in view of this disclosure, to practice the treatment methods of the present technology.

In one embodiment, the TH-302 or another compound of formula I is provided in 100 mg vials, lyophilized, and dissolved in D5W and administered intravenously over approximately 30-60 minutes via an infusion pump. The infusion volume depends on the total dose given (in mg) during the infusion. If <1000 mg is being infused, 500 cc of D5W are used for infusion. If the total dose is >1000, 1000 cc of D5W are used for infusion.

The methods of the invention are now described in the context of particular blood cancers.

### Acute Leukemias: ALL and AML

In one embodiment, the blood cancer treated in accordance with the methods of the invention is an acute leukemia. In one embodiment, the acute leukemia is a relapsed or refractory acute leukemia. For treating ALL, AML, other acute leukemias, and other blood cancers, TH-302 or another compound of formula I is administered at a frequency and in amounts described herein. In various embodiments, TH-302 or another hypoxia activated prodrug of formula I is administered as a single agent, i.e., no other drug intended to treat the blood cancer is contemporaneously administered. In various embodiments, TH-302 or another hypoxia activated prodrug of formula I is administered in combination with another anti cancer drug(s). In the combination therapies of the invention, the other drug(s) are administered, in some embodiments, in frequencies and amounts, and via routes, substantially similar if not identical to those conventionally employed.

In one embodiment, the acute leukemia is acute lymphoblastic leukemia (ALL). In one embodiment, the ALL is relapsed or refractory ALL. In one embodiment, the patient is unsuitable for treatment with standard chemotherapy or unwilling to undergo standard chemotherapy. In one embodiment, the patient is Philadelphia chromosome (Ph) positive.

In one embodiment, TH-302 or another hypoxia activated prodrug of formula I is administered in combination with a drug and/or non-drug therapy conventionally used to treat ALL. Examples of such drugs include, without limitation, alemtuzumab (Campath®, Genzyme), amsacrine, asparaginase (also called crisantaspase), cyclophosphamide, cytarabine (ara-C), daunorubicin, doxorubicin (Adriamycin®, Bedford Laboratories), etoposide, mercaptopurine (6-MP), methotrexate, ofatumumab (Arzerra®, GlaxoSmithKline and Genmab), rituximab (Rituxan®, Genentech), teniposide, thioguanine, and vincristine. An example of a suitable non-drug therapy includes, without limitation, radiation and/or bone marrow stem cell transplantation.

In one embodiment, a patient with ALL is treated by administering TH-302 for five consecutive days of a 21 day cycle, and the dose is between 120 and 575 mg/m²/day, including, for example, doses of 180, 240, 340, and 480 mg/m²/day. In some embodiments, the five daily doses are spread out over 8 days.

In one embodiment, the acute leukemia is acute myelogenous leukemia (AML). In one embodiment, the AML is relapsed or refractory AML. In another embodiment, the AML is acute promyelocytic leukemia. In one embodiment, the patient is unsuitable for standard chemotherapy or unwilling to undergo standard chemotherapy. In one embodiment, the patient is Philadelphia chromosome (Ph) positive.

In one embodiment, TH-302 or another hypoxia activated prodrug of formula I is administered in combination with a drug and/or non-drug therapy conventionally used to treat AML. Examples of such drugs include, without limitation, cytarabine, daunorubicin, etoposide, idarubicin, thioguanine, and vinicristine. An example of a suitable non-drug therapy includes, without limitation, radiation and/or bone marrow stem cell transplantation.

In one embodiment, a patient with AML, including but without limitation with promyelocytic leukemia, is treated by administering TH-302 for five consecutive days of a 21 day cycle, and the dose is between 120 and 575 mg/m²/day, including, for example, doses of 180, 240, 340, and 480 mg/m²/day. In some embodiments, the five daily doses are spread out over 8 days.

### Chronic Leukemias: CML, CLL, and Myelofibrosis

In another embodiment, the blood cancer treated is a chronic leukemia. In one embodiment, the acute leukemia is a relapsed or refractory chronic leukemia. For treating CLL, CML, other chronic leukemias, and other blood cancers, TH-302 or another compound of formula I is administered at a frequency and in amounts described herein. In various embodiments, TH-302 or another hypoxia activated prodrug of formula I is administered as a single agent, i.e., no other drug intended to treat the blood cancer is contemporaneously administered. In various embodiments, TH-302 or another hypoxia activated prodrug of formula I is administered in combination with another anti cancer drug(s). In the combination therapies of the invention, the other drug(s) are administered, in some embodiments, in frequencies and amounts, and via routes, substantially similar if not identical to those conventionally employed.

In one embodiment, the chronic leukemia is chronic myelogenous leukemia (CML). In one embodiment, the CML is relapsed or refractory CML. In one embodiment, the CML is in accelerated or blast phase and/or is unsuitable for treatment with for treatment with standard chemotherapy or unwilling to undergo standard chemotherapy.

In one embodiment, TH-302 or another hypoxia activated prodrug of formula I is administered in combination with a drug and/or non-drug therapy therapy conventionally used to treat CML. Examples of such drugs include, without limitation, tyrosine kinase inhibitors, including but not limited to imatinib, dasatinib, and nilotinib, hydroxyurea, interferon-α 2b, and busulfan. An example of a suitable non-drug therapy includes, without limitation, radiation and/or bone marrow stem cell transplantation.

In one embodiment, a patient with CML is treated by administering TH-302 for five consecutive days of a 21 day cycle, and the dose is between 120 and 575 mg/m²/day, including, for example, doses of 180, 240, 340, and 480 mg/m²/day. In some embodiments, the five daily doses are spread out over 8 days.

In one embodiment, the chronic leukemia is chronic lymphocytic leukemia (CLL). In one embodiment, the CLL is relapsed or refractory CLL. In one embodiment, the patient is unsuitable for treatment with standard chemotherapy or unwilling to undergo standard chemotherapy.

In one embodiment, TH-302 or another hypoxia activated prodrug of formula I is administered in combination with a drug and/or non-drug therapy conventionally used to treat CLL. Examples of such drugs include without limitation, alemtuzumab, bendamustine, chlorambucil, cyclophosphamide, fludarabine, and rituximab. An example of a suitable non-drug therapy includes, without limitation, radiation and/or bone marrow stem cell transplantation.

In one embodiment, a patient with CLL is treated by administering TH-302 for five consecutive days of a 21 day cycle, and the dose is between 120 and 575 mg/m²/day, including, for example, doses of 180, 240, 340, and 480 mg/m²/day. In some embodiments, the five daily doses are spread out over 8 days.

In one embodiment, the chronic leukemia is myelofibrosis (MF). In one embodiment, the myelofibrosis is advanced myelofibrosis. In one embodiment, the MF is relapsed or refractory MF. In one embodiment, the myelofibrosis (whether or not diagnosed and/or prior treated) is characterized by the patient having one or more of (1) hemoglobin concentration of less than 10 g/dL, (2) platelet count of less than 100×10⁹/L and/or white blood cell count of less than 4×10⁹/L or greater than 30×10⁹/L, and (3) splenomegaly greater than or equal to 10 cm below left costal margin. In one embodiment, the patient is unsuitable for treatment with standard chemotherapy or unwilling to undergo standard chemotherapy.

In one embodiment, TH-302 or another hypoxia activated prodrug is administered in combination with a drug and/or non-drug therapy conventionally used to treat MF. Examples of such drugs include, without limitation, hydroxyurea. An example of a suitable non-drug therapy includes, without limitation, radiation and/or bone marrow stem cell transplantation.

### Myelodisplastic Syndrome

In one embodiment, the blood cancer treated in accordance with the methods of the invention is myelodysplastic syndrome (MDS). In one embodiment, the MDS is high risk MDS, which means that the patient has a higher likelihood of developing AML, compared to MDS that is not high risk. In one embodiment, the high risk MDS is a relapsed or refractory chronic myelomonocytic leukemia (CMML) characterized by greater than 5% bone marrow blasts. In one embodiment, the MDS is a refractory anemia characterized, using world health organization (WHO) classification, by excess blasts RAEB-1 or RAEB-2. For treating MDS and other blood cancers, TH-302 or another compound of formula I is administered at a frequency and in amounts described herein. In various embodiments, TH-302 or another hypoxia activated prodrug of formula I is administered as a single agent, i.e., no other drug intended to treat the blood cancer is contemporaneously administered. In various embodiments, TH-302 or another hypoxia activated prodrug of formula I is administered in combination with another anti cancer drug(s). In the combination therapies of the invention, the other drug(s) are administered, in some embodiments, in frequencies and amounts, and via routes, substantially similar if not identical to those conventionally employed.

In one embodiment, TH-302 or another hypoxia activated prodrug of formula I is administered in combination with a drug and/or non-drug therapy conventionally used to treat MDS. Examples of such drugs include, without limitation, cytarabine, idarubicin, fludarabine, topotecan, hypomethylating agents such as azacytidine and decitabine, immunomodulating agents such as lenalidomide and thalidomide, and immunosuppression agents such as anti-thymocyte globulin (ATG) and cyclosporine. An example of a suitable non-drug therapy includes, without limitation, radiation and/or bone marrow stem cell transplantation.

In one embodiment, a patient with MDS is treated by administering TH-302 for five consecutive days of a 21 day cycle, and the dose is between 120 and 575 mg/m²/day, including, for example, doses of 180, 240, 340, and 480 mg/m²/day. In some embodiments, the five daily doses are spread out over 8 days.

### Multiple Myeloma

In one embodiment, the blood cancer treated in accordance with the methods of the invention is multiple myeloma (MM). In one embodiment, the MM is refractory or relapsed MM, including but not limited to lenalidomide or thalidomide refractory MM and bortezomib refractory MM. For treating MM and other blood cancers, TH-302 or another compound of formula I is administered at a frequency and in amounts described herein. In various embodiments, TH-302 or another hypoxia activated prodrug of formula I is administered as a single agent, i.e., no other drug intended to treat the blood cancer is contemporaneously administered.

In one embodiment of the methods of the invention to treat MM and other blood cancers, the TH-302 or another compound of formula I is administered on days 1, 4, 8, and 11 of a 21 day cycle, and the dose is between 120 and 575 mg/m²/day, including, for example, doses of 180, 240, 340, and 480 mg/m²/day.

In various embodiments, TH-302 or another hypoxia activated prodrug of formula I is administered in combination with another anti cancer drug(s). In the combination therapies of the invention, the other drug(s) are administered, in some embodiments, in frequencies and amounts, and via routes, substantially similar if not identical to those conventionally employed.

In one embodiment, TH-302 or another hypoxia activated prodrug of formula I is administered in combination with a drug and/or non-drug therapy conventionally used to treat MM. Examples of such drugs include without limitation, bortezomib, carmustine, cyclophosphamide, dexamethasone, doxorubicin, idarubicin, lenalidomide, melphalan, prednisone, thalidomide, and vincristine. An example of a suitable non-drug therapy includes, without limitation, radiation and/or bone marrow stem cell transplantation

In one embodiment, TH-302 or another compound of formula I is administered as a combination therapy with bortezomib to treat MM, including relapsed or refractory forms of this disease, including but not limited to patients who have failed prior bortezomib and/or lenalidomide (or thalidomide) therapy. In one embodiment, the TH-302 or another compound of formula I is administered on days 1, 4, 8, and 11 of a 21 day cycle, and the dose is between 120 and 575 mg/m²/day, including, for example, doses of 180, 240, 340, and 480 mg/m²/day. The bortezomib is administered as commercially supplied at the FDA approved doses of 1.0 mg/m²/day or 1.3 mg/m²/day on the same cycle. In one embodiment, TH-302 is administered at least 30 minutes to 4 hours, i.e., at least 2 hours, before the bortezomib. See PCT Pub. No. 2010/048330, incorporated herein by reference.

In one embodiment, TH-302 is administered as a combination therapy with lenalidomide and/or dexamethasone to treat multiple myeloma, including relapsed or refractory forms of this disease, including but not limited to patients who have failed bortezomib and/or lenalidomide (or thalidomide) therapy. In one embodiment, the TH-302 or another compound of formula I is administered on days 1, 4, 8, and 11 of a 21 day cycle, and the dose is between 120 and 575 mg/m²/day, including, for example, doses of 180, 240, 340, and 480 mg/m²/day. In this embodiment, the lenalidomide is administered as commercially supplied at the FDA approved dose of 25 mg on days 1 to 14, and dexamethasone is administered as commercially supplied at the FDA approved dose of 40 mg on days 1-4 and 9-12 of the same cycle. In another embodiment, the TH-302 is administered on days 1, 4, 8, 11, 15, 18 of a 28 day cycle, and the dose is between 120 and 575 mg/m²/day, including, for example, doses of 180, 240, 340, and 480 mg/m²/day; the lenalidomide is administered as commercially supplied at the FDA approved dose of 25 mg on days 1 to 21; and the dexamethasone is administered as commercially supplied at the FDA approved dose of 40 mg on days 1-4, 9-12 and 17-20 of the same cycle.

### Hypoxic Markers

In various embodiments of the invention, a marker of hypoxia is used to select patients for treatment and/or to identify patients that are responding (or not responding) to therapy. Hypoxia markers have been developed in the course of studies showing that hypoxia promotes more aggressive solid tumor phenotypes and associates with resistance to radiation and many chemotherapies, as well as likelihood of tumor invasion and poor patient survival. In particular, cells at pO₂ <10 mm Hg resist the ionizing effect of radiotherapy and cytotoxic effect of chemotherapy. Hypoxic necrotic foci with pseudopalisading tumor cells are one of the features that define glioblastoma (GBM), for example. Thus, a variety of methods have been devised to assess degree of hypoxia in xenografts and patient tumors, and, in accordance with the invention, these methods, suitably modified and practiced as described herein, are used in certain embodiments of the methods of the invention to select patients and assess response to therapy. In general, the invention provides methods for identifying patients suitable for therapy with a hypoxia activated prodrug in which a marker of hypoxia is used to identify that a patient's cancer is hypoxic and then the patient is treated with a hypoxia activated prodrug, i.e., the higher the degree of hypoxia, the more likely the patient will respond to therapy with a hypoxia activated prodrug. Those of skill in the art will appreciate, in view of this disclosure, that these methods are useful in all cancers, not just blood cancers.

Traditionally, the gold standard for measuring hypoxia has been the use of a polarographic oxygen-sensitive probe, which provides direct measurement of tissue oxygen tension. However, this method has limitations, such as its inability to differentiate between viable and necrotic foci, the inaccessibility of many tumor tissues, including those associated with hematologic malignancies of the bone marrow, and the lack of a practical means to apply the technique in large scale. Pimonidazole and EF5, both 2-nitroimidazole compounds, are hypoxia markers that, via immunohistochemical identification of pimonidazole or EF5 protein adducts, can give a reliable estimate of radio-biologically relevant hypoxia. Molecular oxygen competes with reducing equivalents in a manner such that pimonidazole (and EF5) binding is effectively inhibited at oxygen concentrations above 14 micromolar. This method reliably identifies viable hypoxic cells specifically (necrotic cells cannot metabolize pimonidazole or EF5).

Other hypoxic markers that have been identified in pre-clinical studies that are suitable for use in accordance with the methods of the invention include GLUT-1, HIF-1a, CA-IX, LDH-A, osteopontin, microRNA markers, including but not limited to miR-210, and VEGF. Each of these proteins or RNAs is up-regulated in hypoxia, and they can be detected by tumor biopsy. More conveniently, however, some of these markers, i.e., CA-IX LDH-A, osteopontin, microRNA markers, including but not limited to miR-210, and VEGF, will be detectable in the blood, serum, or plasma of a patient, allowing a simple blood test, instead of a tumor biopsy, to be used to select patients for hypoxia activated prodrug therapy.

In addition, studies have examined the spatial relationship between tumor hypoxia assessed by immunohistochemistry and [18F]-FDG and [18F]-FMISO autoradiography and PET imaging, and these compounds and similar PET tracers, such as [18F]-EF5, [18F]-FAZA, and [18F]-HX4, can be employed in accordance with the methods of the invention. In addition to autoradiography and PET imaging, MRI imaging of hypoxia, in particular dynamic contrast-enhanced MRI (DCE-MRI), can be used to identify hypoxic cancers and thus identify patients ideal for treatment with hypoxia-activated prodrugs.

Hypoxyprobe®-1 (pimonidazole hydrochloride, marketed by Hypoxyprobe, Inc.) when administered, either IV or orally, is distributed to all tissues in the body including the brain but only forms adducts with proteins in those cells that have an oxygen concentration less than 14 micromolar (equivalent to a pO₂ of 10 mm Hg at 37 degrees Celsius). Hypoxyprobe-1MAb1 is a mouse IgG1 monoclonal antibody that detects protein adducts of Hypoxyprobe-1 in hypoxic cells. This reagent is typically added to each tissue sample. Chromogenic or fluorescent secondary antibody reagents are then used in accordance with the invention to reveal where Hypoxyprobe-1 adducts have formed in the hypoxic tissue.

In addition to these markers of hypoxia, there are other markers that can be used to select patients for hypoxia activated prodrug therapy. The hypoxia activated prodrugs of the invention are activated by reductases, so biopsies or blood tests that show a patient has higher levels of an activating reductase, such as POR (P450 oxido-reductase), MTRR (methionine synthase reductase), and/or NOS (nitric oxide synthase), demonstrate that a patient is more likely to respond to hypoxia activated prodrug therapy. Furthermore, the DNA damage induced by these hypoxia activated prodrugs is repaired by the HDR (also known as HR) system, and the lower the levels of the proteins in this system, including but not limited to BRCA, FANG, XPF (also known as ERCC4), XRCC2 and/or XRCC3, in the blood or tumor biopsy of a patient, the more likely the patient will respond to hypoxia activated prodrug therapy.

Thus, the methods of the invention include methods for determining whether a patient is suitable for or is responding to a therapeutic method of the invention.

The present invention having been described in summary and in detail, is illustrated and not limited by the following examples.

### EXAMPLES

### Example 1

### In Vivo Determination of the Level of Hypoxia in the Bone Marrow

In this example, the hypoxic nature of multiple myeloma was demonstrated by staining the bone marrow of naive and 5T33MM mice with the exogenous hypoxia marker pimonidazole and endogenous hypoxia marker hypoxia inducible factor 1α (HIF1α). The results demonstrate that multiple myeloma cells reside in a more hypoxic bone marrow environment. See also, the reference Hu et al., Blood 116 (9): 1524-1527, 2010, incorporated herein by reference. The effects of TH-302 on multiple myeloma cell lines in vitro were also demonstrated, focusing on apoptosis and cell cycle as well as associated signaling pathways in multiple myeloma. Furthermore, the therapeutic effects of TH-302 in treating multiple myeloma in the 5T33vv mouse were also demonstrated.

Considering the potential role of hypoxia in hematopoiesis and multiple myeloma progression in the bone marrow, the oxygen level in the bone marrow of naive and 5T33MM mice, which mimics the human disease (See, Vanderkerken et al., Immunol Rev. 2003; 194:196-206, incorporated herein by reference), was measured by assessing the exogenous and endogenous hypoxia markers pimonidazole and HIF-1α. Both the exogenous marker pimonidazole and endogenous marker HIF1 α were increased in the bone marrow of 5T33MM mice in contrast to the sporadic and weak positivity of hypoxia markers in the naive mice, demonstrating that a majority of multiple myeloma cells localize in an extensively hypoxic niche. The results demonstrated the hypoxic nature of normal and multiple myeloma bone marrow. See also, Giuliani et al., “Oxygen tension in the bone marrow (BM) of patients with malignant and indolent monoclonal gammopathy: role of hypoxia and hypoxia-inducible factor (HIF)-1α in the regulation of gene expression and pro-angiogenic profiles of CD138+ cells,” Blood. 2009; 114(22):175-176, incorporated herein by reference. Others (Ria et al., “Hypoxia-inducible factor-1 in multiple myeloma progression,” Blood. 2009;114(22): 720 and Azab et al., “Role of hypoxia in the progression and dissemination of multiple myeloma,” Blood. 2009;114(22): 175, each of which is incorporated herein by reference) have reported that there may be a role for hypoxic bone marrow in other blood cancers such as lymphomas and leukemias.

TH-302 induced Go/G1 cell cycle arrest in 5T33vt cells in a hypoxia selective manner. TH-302 induced Go/G1 cell cycle arrest depended on down-regulating cyclin D1/2/3, CDK4/6, p21, p27 and pRb expression. TH-302 triggered specific apoptosis in a dose-dependent manner in LP-1 cells under hypoxia. TH-302 (5 μM) induced apoptosis in LP-1 cells. TH-302 (5 μM) decreased the accumulation of HIF1α in hypoxic RPMI-8226 cells. VEGFa secretion was reduced by TH-302 in 5T33vt cells. *p<0.05, **p<0.01, ***p<0.001, compared to 20% O₂ (n=3).

### Example 2

### In Vitro Testing of TH-302

This example demonstrates that the hypoxic niche of multiple myeloma can also serve as a treatment target. The data demonstrates that the hypoxia activated prodrug, TH-302, exhibits potent, dose dependent in vitro cytotoxicity in multiple myeloma cells with hypoxic selectivity. To demonstrate the growth inhibitory effects of TH-302 on multiple myeloma cells, the cell cycle phase distribution and apoptosis after drug treatment were analyzed. Cell cycle analysis showed that TH-302 induced Go/G1 cell cycle arrest under hypoxic conditions in Karpas-707, LP-1, MMS1, and RPMI-8226 cells. Western blotting further revealed that the effect of TH-302 on cell cycle machinery was mediated by down-regulating cyclin D1/2/3, CDK4/6, p21cip-1, p27kip-1 and pRb expression, whereas CDK2 expression remained undisturbed, as observed in RPMI-8226, LP-1, MMS1 and Karpas-707 multiple myeloma cells. Furthermore, flow cytometry analysis demonstrated that TH-302 induced dose-dependent apoptosis in both human and murine multiple myeloma cells in hypoxic conditions, similar results were also observed in RPMI-8226, 5T33vt, MMS1, Karpas-707 cells. Western blotting further demonstrated that TH-302 activated apoptosis was mediated by down-regulation of the anti-apoptotic proteins BCL-2 and BCL-xL, as well as up-regulation of the expression of cleaved proapoptotic protein caspase-3, 8, 9 and poly ADP-ribose polymerase (PARP). In contrast to the hypoxia-specific toxicity, TH-302 shows very low toxicity in normoxic conditions even at high concentrations, similar results were also found in RPMI-8226, 5T33vt, MMS1, Karpas-707 cells. In addition, it was demonstrated that the production of HIF1α, a regulator of the hypoxic response (Hose et al., “Induction of angiogenesis by normal and malignant plasma cells,” Blood. 2009;114(1):128-143, incorporated herein by reference), decreased with the treatment of TH-302. The expression of HIF1α in a hypoxic condition was reduced following exposure to TH-302 (similar results were also found in 5T33vt, LP-1, Karpas-707 cells), accordingly, the secretion of VEGFa which is a downstream target gene of HIF1α was also significantly decreased. By employing a set of defined gas mixtures (0%, 1%, 1.25%, 1.5%, 2%, 3%, 20% O₂) for drug treatment of the multiple myeloma cells, the oxygen concentration dependent activation of TH-302 was tested in RPMI-8226, LP-1, MMS1 and 5T33vt multiple myeloma cells. The results indicated that, under these test conditions, the threshold of activating TH-302 was<than 1.5% O₂. As the O₂ concentration was reduced to 0%, the fraction of apoptotic cells increased to about 70-75%.

### Example 3

### In Vivo Administration of TH-302 for the Treatment of Multiple Myeloma

Testing TH-302 in the 5T33MMvv mouse demonstrated that in vivo treatment with TH-302 improved many disease parameters. 5T33MMvv mice were treated prophylactically with TH-302 for 3 weeks. From day 1, the following were observed. TH-302 induced significant multiple myeloma cell apoptosis (for TH-302 administered at 12.5 mg/kg, 2.5 fold; at 25 mg/kg, 2.1 fold; and at 50 mg/kg, 3.1 fold), decreased paraprotein secretion (at 12.5 mg/kg, 32% decrease; at 25 mg/kg, 77% decrease; and at 50 mg/kg, 54% decrease), and significantly decreased microvessel density (MVD) (at 12.5 mg/kg, 19% decrease; at 25 mg/kg, 20% decrease; and at 50 mg/kg, 26% decrease) in the bone marrow of treated 5T33MMvv mouse, compared to vehicle-treated 5T33MMvv mice. Therefore, the cancer cytotoxic effect of TH-302 was associated with the hypoxic nature of multiple myeloma cells in the bone marrow. In addition, the data from TH-302 treated naive mice showed no substantial toxicity in terms of body weight, hemoglobin (HGB), red blood cell count (RBC), white blood cell count (WBC), hematocrit (HCT) and microvessel density (MVD), compared to the vehicle-treated naive mice, further indicating the specific hypoxia-activated effect of TH-302 and the limited hypoxia in the normal bone marrow.

### Example 3

### In Vitro Testing of TH-302 in Combination with Bortezomib

This example demonstrates that the combination of TH-302 and bortezomib synergistically induces apoptosis, as evidenced by induced cleavage of poly(ADP-ribose) polymerase and caspase-3/8/9. To further determine the mechanism of induction of apoptosis by this combination, the effect of TH-302, bortezomib and the combination on anti-apoptotic and pro-apoptotic Bcl-2 family proteins using immunoblotting was tested. The results show that pro-apoptotic BH-3 member Noxa and the cleavage of BID were induced by both bortezomib and TH-302. Moreover, the expression of anti-apoptotic Bcl-2 and Bcl-xL was decreased by both TH-302 and bortezomib; however, anti-apoptotic Mcl-1 accumulated with bortezomib but decreased with TH-302, indicating that TH-302 can overcome the resistance to bortezomib via targeting Mcl-1.

### Example 4

### In Vivo Administration of TH-302 in Combination with Bortezomib for the Treatment of Multiple Myeloma

The combination of TH-302 and Bortezomib tested in the 5T33MMvv mouse model demonstrates that in vivo combination treatment showed impressive improvements in multiple disease parameters, induced significant decreased tumor burden, paraprotein secretion and microvessel density (MVD), compared to TH-302 or bortezomib-alone treated 5T33MMvv mice (p<0.01)

Taken together, the results demonstrate that multiple myeloma cells reside in an extensively hypoxic bone marrow microenvironment. Hypoxia-activated treatment with TH-302 as a monotherapy shows efficacy in treatment of multiple myeloma both in vitro and in vivo. The findings in this test indicate that targeting the hypoxic bone marrow niche provides a useful and novel treatment strategy for multiple myeloma and other blood cancers, where the hypoxic region of the bone marrow can lead to the formation of cancer stem cells and various blood cancers.

### Example 5

### Clinical Administration of TH-302 for the Treatment of Advanced Leukemia

Clinical investigations were conducted to determine the safety, tolerability and clinically relevant disease responses of TH-302 in patients with acute leukemias, advanced phase chronic myelogenous leukemia (CML), high risk myelodysplastic syndromes, advanced myelofibrosis or relapsed/refractory chronic lymphocytic leukemia (CLL). TH-302 was administered as a 30 to 60 minute intravenous infusion daily for 5 days followed by 2 weeks off therapy. As needed, the 5 days of dosing could be spread out over 8 days. Patients who successfully completed a 3-week treatment cycle without evidence of significant treatment-related toxicity or progressive disease were continued on treatment and could receive treatment for up to six cycles.

Twenty-one patients have been treated in the study at TH-302 daily doses ranging from 120 mg/m² to 460 mg/m². All subjects had either AML or ALL and had generally received multiple prior therapies for their disease prior to enrolling for the study. Two patients with AML treated with TH-302 at the 120 mg/m²/day dose had stable disease, including one patient who had their dose escalated to 170 mg/m²/day after Cycle 2 when their peripheral blast count decreased after each cycle of TH-302 while their platelet count was gradually improving.

One patient with ALL treated with TH-302 at the 170 mg/m²/day dose had a partial response based on the normalization of the bone marrow blast count as measured by bone marrow biopsy after cycle 1. One patient with AML and one patient with ALL treated with TH-302 at the 240 mg/m²/day dose had stable disease after cycle 1. One patient with ALL treated with TH-302 at the 330 mg/m²/day dose had stable disease after cycle 1.

These results show that the methods of the invention are effective in treating blood cancers.

While certain embodiments have been illustrated and described in the foregoing examples, it will be understood that changes and modifications can be made in the foregoing methods to practice the present technology in accordance with ordinary skill in the art without departing from the present technology in its broader aspects as defined in the following claims.

The inventions have been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

1. A method of treating a blood cancer comprising administering a therapeutically effective amount of a hypoxia activated prodrug selected from the group consisting of TH-281, TH-302, and TH-308 to a patient in need of such treatment thereby treating the cancer.

2. The method of claim 1, wherein the blood cancer treated is selected from the group consisting of multiple myeloma, an acute leukemia, an advance phase chronic myelogenous leukemia (CML), a high risk myelodysplastic syndrome (MDS), an advanced myelofibrosis (MF), or a relapsed or refractory chronic lymphocytic leukemia (CLL).

3. The method of claim 2, wherein the blood cancer treated is an acute leukemia that is either a relapsed or a refractory acute leukemia.

4. The method of claim 2, wherein the blood cancer treated is an acute leukemia that is either relapsed or refractory acute lymphoblastic leukemia (ALL) or relapsed or refractory acute myelogenous leukemia (AML).

5. The method of claim 1, wherein the patient is unsuitable for standard chemotherapy.

6. The method of claim 1, wherein the hypoxia activated prodrug administered is TH-302.

7. The method of claim 6, wherein TH-302 is administered as single agent therapy for five consecutive days, or five of eight consecutive days, of a 21 day cycle, and the therapeutically effective amount is 120 mg/m²/day to 575 mg/m²/day.

8. The method of claim 6, wherein TH-302 is administered as single agent therapy to treat multiple myeloma, on days 1, 4, 8, and 11 of a 21 day cycle, and the therapeutically effective amount is 120 mg/m²/day to 575 mg/m²/day.

9. The method of claim 6, wherein TH-302 is administered as a combination therapy further comprising bortezomib to treat multiple myeloma, on days 1, 4, 8, and 11 of a 21 day cycle, and the therapeutically effective amount is 120 mg/m²/day to 575 mg/m²/day.

10. The method of claim 6, wherein TH-302 is administered as a combination therapy further comprising lenalidomide and dexamethasone to treat multiple myeloma, on days 1, 4, 8, and 11 of a 21 day cycle, and the therapeutically effective amount is 120 mg/m²/day to 575 mg/m²/day.

11. The method of claim 6, wherein TH-302 is administered as a combination therapy further comprising lenalidomide and dexamethasone to treat multiple myeloma on days 1, 4, 8, 11, 15, 18 of a 28 day cycle, and the therapeutically effective amount is 120 mg/m²/day to 575 mg/m2/day.
